(19) United States
(12) Patent Application Publication
(10) Pub. No.: US 2023/0248642 A1
(43) Pub. Date: Aug. 10, 2023

(54) INJECTABLE HIGH-DRUG-LOADED NANOCOMPOSITE GELS AND PROCESS FOR MAKING THE SAME

(71) Applicant: NUECOLOGY BIOMEDICAL INC., Richmond (CA)

(72) Inventors: Chung Chin SUN, Richmond (CA); Dean Mo LIU, Surrey (CA)

(73) Assignee: NUECOLOGY BIOMEDICAL INC., Richmond, BC (CA)

(21) Appl. No.: 18/015,495

(22) PCT Filed: Jul. 12, 2021
(86) PCT No.: PCT/CA2021/050955

§ 371 Date: Jan. 10, 2023

Related U.S. Application Data
(60) Provisional application No. 63/050,396, filed on Jul. 10, 2020.

(51) Int. Cl. A61K 9/00; A61K 31/337; A61K 9/51; C07K 16/22; C07K 16/28; C07K 16/42; C07K 16/30; C07K 16/24; A61K 31/07; A61K 31/355; A61K 31/4745; A61K 31/704; A61K 31/12

(52) U.S. Cl. CPC A61K 9/0019; A61K 31/337; A61K 9/5161; C07K 16/22; C07K 16/2803; C07K 16/42; C07K 16/30; C07K 16/241; C07K 16/2887; A61K 31/07; A61K 31/355; A61K 31/4745; A61K 31/704; A61K 31/12; C07K 16/2863; C07K 2317/24; C07K 2317/21

(57) ABSTRACT

This invention discloses an injectable nanocomposite gel composition and the method of making the composition. The composition is composed of amphiphilic alginate nanoparticle, gel stabilizer, gel crosslinker, and gel structural modifiers. The nanocomposite gel can be manufactured into a form of highly-viscous gel or a solid-like gel, used as a vehicle to carry and deliver pharmaceutically active ingredients with high drug load via injection administration for medical uses.

### FIELD OF THE INVENTION

This invention discloses an injectable nanocomposite gel composition and the method of making the composition, which can be used as a vehicle to carry and deliver an active ingredient.

### BACKGROUND OF THE INVENTION

Injectable hydrogels have been considerably reported over decades in literature for a number of biomedical applications ranging from fillers, implantable vehicles, carrier for drugs, cells, and supplements, etc. Natural polysaccharides such as chitosan, alginates, hyaluronates, glycan, dextran, etc. have been received large attention in synthesis of specific hydrogel for medical application, due to their excellent biocompatibility, biodegradability, processability, and ease of chemical modification. Therefore, use of natural polysaccharides, either as a primitive form or as a modified form, such as hydrophobically-modified or amphiphilically-modified, had received enormous interests for medical uses. For drug delivery application, such modified version is able to form nano-size particles which can be used to entrap pharmaceutically active ingredients of different physicochemical properties (e.g., hydrophobic and hydrophilic properties) simultaneously, followed by controlled delivery, via vein administration, intramuscular injection, intraperitoneal injection or subcutaneous injection to the host for therapeutic purpose.

Injection of hydrogel will lead to the formation of a “depot” at the site of administration that slowly and continuously releases the drug to the tumor or diseased site and its surrounding tissue. This kind of injectable gel for physical targeting provides a number of advantages over passive or other actively targeted therapies in that it can deliver a drug throughout the tumor or diseased sites regardless of vascular status and/or biological environment surrounding the site of administration, thus providing accurate dosing without systemic toxicity or due to possible variants between genders, ages, and races. For instance, poloxamer gels have been widely applied in drug delivery since they are relatively easy to manufacture and already widely employed in the pharmaceutical industries as “generally regarded as safe” (GRAS) excipients. This type of hydrogels mainly focuses on poloxamer 407. For localized cancer therapy, intratumoral, peritumoreal, and intravesical injection of such type of hydrogel composed of Pluronic® F127 (F127) has been reported (Y. L. Lo, C. Y. Hsu, H. R. Lin, pH- and thereto-sensitive pluronic/poly(acrylic acid) in situ hydrogels for sustained release of an anticancer drug, J Drug Target, 21 (2013) 54-66). However, such poloxamer gels for drug delivery applications have substantial drawbacks including the gelation time being too long, poor stability, poor mechanical properties and short residence times due to rapid dissolution once placed in a biological environment.

US 20120100103 discloses an in situ-forming injectable hydrogel comprising two or more homogeneous or heterogeneous polymers, which are bonded to each other by a dehydrogenation reaction between phenol or aniline moieties on adjacent polymers. US 20140065226 provides compositions including an environmentally-responsive hydrogel and a biocompatible monomer or polymer including an amino acid side chain (i.e., having an amino acid linked to the remainder of the monomer or polymer through its side chain), which has environmentally-responsive behavior at physiological condition, such as temperature and is useful as injectable and topical formulations, particularly for biomedical applications such as so localized drug delivery.

US20150366975A1 discloses a thermosensitive injectable hydrogel based on hyaluronic acid and a copolymer of polyethylene oxide (PEO) and polypropylene oxide (PPO), which has a gel formation temperature from 30° C. to 37° C. The thermosensitive injectable hydrogel provides a potential drug delivery system that can increase therapeutic efficacy of the drug.

It is desirable to develop a new drug delivery system used for injection administration.

### SUMMARY

Accordingly, the present invention provides a new approach to deliver one or more active ingredients or drugs in humans by combining such amphiphilic nanoparticles with a self-sustained porous matrix phase to form a drug-carrying injectable nanocomposite hydrogel in either highly-viscous or solid form for a variety of medical uses, for example for anti-tumor treatment.

The present invention generally relates to an injectable nanocomposite gel composition and the method for preparing the same. In particular, the present invention relates to an injectable hydrogel.

In one aspect, the present invention provides a composition of injectable nanocomposite gel, which comprises an amphiphilic alginate nanoparticle, a hyaluronic salt or derivative, an alginate salt or derivative, and an ionic crosslinker.

In one embodiment of the invention, the composition further comprises an active ingredient. The active ingredient is selected from the group consisting of an antibody drug, a biosimilar drug, a protein-like drug, a chemo-drug, and the combination thereof.

In other embodiment of the invention, the active ingredient is selected from the group consisting of trastuzumab, bevacizumab, gemtuzumab, inotuzumab, polatuzumab, sacituzumab, adalimumab, infliximab, rituximab, and the combinations thereof.

In one embodiment of the invention, the active ingredient is a water-insoluble active ingredient, which is selected from the group consisting of vitamin A and its derivatives, Vitamin E and its derivatives, paclitaxel, docetaxol, camptothecin, doxorubicine, and curcumin.

In one example of the invention, the amphiphilic alginate has a molecular weight of 5,000 g/mole to 50,000 g/mole. The alginate salt is sodium alginate and has a molecular weight of 10,000 g/mole to 60,000 g/mole.

In one example of the invention, the hyaluronate is a hyaluronic salt and has a molecular weight of 100,000 g/mole to 1,000,000 g/mole, preferrably 100,000 g/mole to 500,000 g/mole.

In one example of the invention, the ionic crosslinker is selected from the group consisting of CaCl₂), CaCO₃, calcium phosphates, ZnCl₂, BaCl₂, and the mixture thereof. The gross concentration of the ionic crosslinker is from 0.5% to 5% (on gel weight base).

In one example of the present invention, the amphiphilic alginate nanoparticle is a fatty acid-conjugated alginate. The fatty acid-conjugated alginate is selected from the group consisting of oleic acid-conjugated alginate, stearic acid-conjugated alginate, linoleic acid-conjugated alginate, palmitic acid-conjugated alginate, and the combinations thereof. Preferably, the amphiphilic alginate nanoparticle is oleic acid-conjugated alginate.

According to the invention, the amphiphilic alginate-based nanoparticle can be used either alone or in combination with second drug being encapsulated in said amphiphilic alginate nanoparticle and allowing the composition to form a solid-like gel or high-viscous gel by crosslinking via the addition of metallic salts.

In another aspect, the present invention provides an injectable nanocomposite gel comprising an amphiphilic alginate-based nanoparticle and a salt of alginate and a hyaluronate, and a active ingredient and an ionic crosslinker or a mixture of the ionic crosslinkers.

In an embodiment of the invention, a low-molecular-weight alginate-based macromolecule is formed from an amphiphilic alginate or its derivatives (developed by Nuecology Biomedical Inc. Richmond, BC, Canada). According to the invention, the amphiphilic alginate is able to self-assemble into a nano-sized spherical nanoparticle in an aqueous environment which can be applicable to encapsulate hydrophobic In one specific example of the invention, amphiphilic alginate is a fatty-acid-conjugated alginate, and the active agent is a hydrophilic drug. T.

According to the invention, the said amphiphilic alginate nanoparticle can be used either alone or carries with a hydrophobic drug, further combining with gel matrix to ultimately develop a nanocomposite gel after gelation, where the final gel entity can be used for a subsequent injection to a subject for anti-tumor treatment. This fatty-acid-conjugated alginate nanoparticle exhibited excellent biocompatibility, drug loading ability and cellular uptake efficiency.

In a preferred embodiment of the present invention, the amphiphilic alginate can be used alone or in combination with an active ingredient, either water-soluble or water-insoluble, if practically needed, combined with highly porous gel matrix, to form a drug-carrying injectable nanocomposite gel. The porous gel matrix carried a water-soluble drug, which is used for specific anti-tumor treatment and the drug in the porous gel matrix can be a protein, an antibody drug, a biosimilar drug, an RNA-based molecule included but not limited to RNAi, microRNA, etc.

According to the invention, the porous gel matrix is composed of (1) a gel modifier, which included mid-to-high-molecular weight hyaluronate salts or its derivatives, (2) a gel former, which included low-molecular weight alginate salts in combination with low-molecular weight amphiphilic alginates, where the amphiphilic alginate is more preferable to have a cytotoxic potency to particularly cancerous cells or tissues, but is compatible to normal cells or tissues, (3) a gel stabilizer, included calcium chloride, (4) a gel crosslinker, which included but not limited to calcium chloride, calcium carbonate, barium chloride or zinc chloride, or metallic salts with divalent or trivalent coordination to those gel forming ingredients. The manufacturing procedures to produce resulting nanocomposite hydrogel are given below.

In another preferred embodiment, this invention provides the steps of:
(1) preparing a mixture of alginate-based solution as Solution (1), comprising an alginate salt and an amphiphilic alginate nanoparticle at the ratio ranging from 1:1 to 10:1;
(2) mixing a hyaluronate with a metallic salt to obtain a mixture as Solution (2);
(3) mixing Solution (1) and Solution (2) at the ratio (by weight) ranging from 0.5:1 to 5:1 to obtain a homogeneous nanocomposite gel.

According to the invention, the gel composition is used for drug delivery use. The said injectable gel was prepared by the method of the steps:

(i) preparing a mixture of alginate-based solution as Solution (1), where alginate salts and amphiphilic alginate with preferred weight ratio is prepared;
(ii) preparing a mixture of hyaluronates and metallic salts solution as Solution (2);
(iii) by mixing Solution (1) and Solution (2) with a ratio (by weight) from 0.5:1 to 2:1.

In one example of the invention, a high-viscous or solid-like gels can then be prepared by mixing Solution (1) with Solution (2), with gelation occurred in a manageable time period, to form a homogeneous nanocomposite gel. While adding biosimilar, antibody or protein drug, the drug was first dissolved and mixed in Solution (1) with a concentration ranging (in terms of final concentration in injectable gels) from 1.0% to 15% by weight, to form Solution (3). After then, by mixing Solution (2) and Solution (3), under continuous stirring, a final solid-like injectable gel can be formed for a subsequent medical uses.

The features and advantages of the present invention will be apparent to those skilled in the art. While numerous changes may be made by those skilled in the art, such changes are within the scope of this invention.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

FIG. 1 shows the viscosity changes with angular frequency for both drug-free nanocomposite gel and trastuzumab-carrying gel.

FIG. 2 shows the time-dependent variation of G and G′ under consecutive on-off shear load, where the nanocomposite gel shows a rapid structural restoration, i.e., self-healing behavior, after shear load is removed.

FIG. 3 shows the influence of ionic crosslinker on the G and G′ of the nanocomposite gel, where the G, storage modulus, remained sufficiently high for lower Ca concentration, but higher Ca deteriorates considerably the G′, loss modulus.

FIG. 4 shows the release profile of biosimilar drug, trastuzumab, in a concentration range of 2.5%, 5%, and 10%, eluting from the trastuzumab gel in-vitro, which shows a fast release at first 48 hours, followed by a slow release to 168 hours, suggesting a 7-day release can be manageable and optimized.

FIG. 5 shows the cytotoxicity study for the trastuzumab (T-mAb) gel with different T-mAb concentration and other controlled protocols, where the cytotoxic data shows a promising outcome for the gel to kill highly malignant breast cancer SKBR3 cells.

FIG. 6 shows that highly porous gel structure was microscopically observed for both nanocomposite gels with and without loading drug. The porous structure facilitates drug release and also can be tuned for a controllable degradation profile when injected into a biological host.

FIG. 7 shows the histopathological analysis of the mice after a 14-day acute toxicity test using nanocomposite gel subcutaneously injected on the right flank region of the mice, where no significant lesion was measurable after the test, indicating a biosafety of the gel disclosed in this invention.

FIG. 8 shows the preparation procedures for the formation of pure AGO injectable gel (Sample (A)), and dual-drug-carrying AGO injectable gel (PTX-T-mAb gel, Sample (B)), where both types of injectable gels were successfully fabricated.

FIG. 9 shows the cell viability of the SKBR-3 cells in terms of free paclitaxel-T-mAb (in solution form, termed as “Free PTX”) and PTX-T-mAb (in gel form, termed as “PTX gel”), where the paclitaxel has a range of concentrations from 0.25 ug/mL to 4 ug/mL, and T-mAb has a concentration of 0.025 ug/mL to 0.4 ug/mL in the both samples.

FIG. 10 shows the growth profile of the SKBR-3 derived breast tumor in mice with co-delivery of paclitaxel chemo-drug and T-mAb Biosimilar drug in form of solution form and gel form. A co-release of both drugs from injectable gel with sufficient drug concentration ensures a synergistic efficacy against the growth of breast tumor to a considerable extent.

### DETAILED DESCRIPTION

It is to be understood that, if any prior art publication is referred to herein, such reference does not constitute an admission that the publication forms a part of the common general knowledge in the art, in any countries or regions.

For the purpose of this specification, it will be clearly understood that the word “comprising or composed of” means “including but not limited to”, and that the word “comprises” has a corresponding meaning.

Unless defined otherwise, all technical and scientific terms used herein have the meaning commonly understood by a person skilled in the art to which the present invention belongs. One skilled in the art will recognize many methods and materials similar or equivalent to those described herein, which could be used in the practice of the present invention. Indeed, the present invention is in no way limited to the methods and materials described.

According to the invention, an anibody or biosimlar or protein-like drug with high payload can be encapsulated by the said nanocomposite gel where drug potency can be enhanced to a large extent than that of free drug to against highly maligant tumor, take breast tumor as one exemplary case, under the same controlled protocol, and the drug-carrying injectable gel can be prepared in a specific and facile manner of production.

According to the invention, a vaccine with high payload can be encapsulated by the said nanocomposite gel where the vaccine efficacy can be enhanced to a large extent than that of vaccine alone to induce an immune response to recognize and fight against infective diseases, wherein the vaccine includes but not limited to whole pathogen vaccines, subunit vaccines, nucleic acid vaccines, and viral vectored vaccines.

Therefore, the present invention provides an antibody (or interchangeably, biosimilar as disclosed in this invention) drug-containing injectable gel, which includes a water-soluble active ingredient selected from the group comprising of trastuzumab, bevacizumab, gemtuzumab, inotuzumab, polatuzumab, sacituzumab, adalimumab, infliximab, and rituximab, a pharmaceutically acceptable biosimilar or interchangeably antibody drug derivative, either alone or in combination with a second water-insoluble active ingredient, comprising paclitaxel, docetaxel, doxorubicin, and curcumin, encapsulated in said amphiphilic alginate nanoparticle.

In some embodiments, the active ingredient is biosimilar drug or its derivatives.

According to the present invention, the amphiphilic alginate nanoparticles have hydrophobic and hydrophilic moieties to respectively interact with hydrophobic and hydrophilic molecules. The amphiphilic alginate carrier may include fatty-acid-conjugated alginate and/or derivatives thereof. Examples of said fatty-acid-conjugated alginate and derivatives thereof include, but are not limited to, oleic acid-conjugated alginate, stearic acid-conjugated alginate, linoleic acid-conjugated alginate, cholesterol-modified alginate. In an exemplary embodiment, the amphiphilic alginate-based nanoparticle is oleic acid-modified alginate.

According to the present invention, the antibody drug-containing injectable nanocomposite gel may use alone or further include an additional pharmaceutically active ingredient that is carried by the amphiphilic alginate nanoparticle. Examples of the additional active ingredient if pharmaceutically required, which is also water-insoluble includes, but are not limited to, Vitamin A and its derivatives, Vitamin E and its derivatives, anti-cancer drugs such as paclitaxel, docetaxol, camptothecin, doxorubicine, etc. According to the present invention, the said amphiphilic alginate nanoparticle has a particle size that ranges from 50 nm to 700 nm. In some embodiments, the said amphiphilic alginate nanoparticle has a particle size that ranges from 50 nm to 350 nm.

In addition, the present invention provides a method for anticancer drug in a subject, which includes administering to the subject the pharmaceutical composition by injection route described in this invention.

The pharmaceutical composition according to the present invention can be formulated into a dosage form suitable for injection administration using technology well known to those skilled in the art, which includes, but is not limited to, subcutaneous injection, intramuscular injection, intratumoral injection, and intraperitoneal injection.

The injectable nanocomposite gel according to this invention where the amphiphilic alginate nanoparticle plays a route not only capable of carrying a second pharmaceutically active ingredient if practically needed, which can be water-insoluble, but also acting as a buffer to accommodate the gelation rate of the injectable gel when the said Solution (2) and Solution (3) aforementioned were mixed. That is to say, the gelation time when those two solutions may become longer, from seconds to minutes or even prolonged, to ensure a final nanocomposite gel to be physically and chemically homogeneous for a subsequent use.

The invention will be further described by way of the following examples. However, it should be understood that the following examples are solely intended for the purpose of illustration and should not be construed as limiting the invention in practice.

### EXAMPLES

### Example 1 Preparation of Injectable Nanocomposite Gel

1.1 Step 1

Solution (1) was prepared by mixing the gel stabilizer and/or crosslinker with structural modifier (hyaluronate salts which is employed to modify viscosity and homogenization of the resulting solution) into a first liquid medium.

1.2. Step 2

Solution (2) was prepared by mixing the amphiphilic alginates and alginate salts into a second liquid medium, which were acting as a dual-function ingredient for both gel former and drug carrier if practically required.

1.3. Step 3

Further mixing Solution (1) and Solution (2), stirring constantly, see below Drawing, to form a final nanocomposite hydrogel with a gelation time (from a viscous liquid to form a solid-like gel) ranging from 0.5 minutes to 20 minutes, depending on the concentration of CaCl₂, CaCO₃, ZnCl₂, or BaCl₂, as ionic crosslinker and the said gel former, i.e., amphiphilic alginate nanoparticles. The ionic crosslinker with a concentration of 0.5-5 wt % was used to form the injectable nanocomposite gel and the said amphiphilic alginate nanoparticle with a concentration of 0.05-2.0 wt %. The higher concentration of the amphiphilic alginate nanoparticle in said gel composition, the longer time period, for instance, from seconds to minutes or prolonged duration as increasing the amount of such amphiphilic nanoparticles, upon solid-gel development.

### Example 2 Viscosity Changes with Angular Frequency

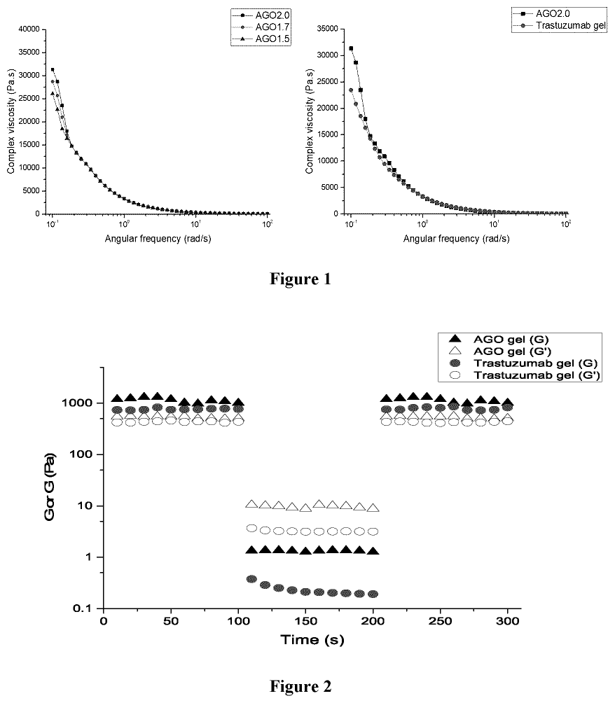

It is also important to learn the resulting injectable nanocomposite hydrogel can be prepared into a solid-like gel in both drug-free gel and trastuzumab-carrying gel (trastuzumab concentration is 10 wt % on weight base of the gel), where the gel viscosity is decreased significantly with increasing strain frequency, as shown in FIG. 1 and the AGO2.0 represents the gel is composed of amphiphilic alginate nanoparticle 0.1 wt % and alginate 2.0 wt %, AGO1.7 represents amphiphilic alginate nanoparticle 0.3 wt % and alginate 1.7 wt %, and AGO1.5 represents amphiphilic alginate nanoparticle 0.5 wt % and alginate 1.5 wt %, while the rest ingredients kept the same.

The lower the gel viscosity under higher angular frequency is able to translate to a condition resemble that of syringe injection, which means the said nanocomposite gel and trastuzumab gel show shear-thinning behavior and allow to be injectable.

### Example 3 Self-Healing Property of the Gels

A shear-dependent storage modulus (G) and loss modulus (G′) is given in FIG. 2, where the both drug-free gel and trastuzumab gel were subjecting to shear for 100 seconds and no shear for an alternative 100 seconds. While subjecting to shear force, G and G′ were decreased to a considerably low level (time period from 100 to 200 seconds), and after removal of the shear (200-300-second period), the G and G′ restored to original status (0-100-second period) for both gels. This can be explained in terms of gel structure variation where the gel structure was disrupted considerably while subjecting to shear force, and the structure restored to almost completely as the one at initial shear-free state right after the shear force removed. This is then able to consider as a self-healing property of the gels prepared and disclosed in this invention, even in the presence of high-load trastuzumab gel, i.e., 10 wt %. One alternative advantage of such a structural restoration or self-healing behavior of the said gel is that since structural integrity will influence a subsequent drug diffusion throughout the gel, restoration of the gel structure found in this invention ensures, to some extent, the same or similar drug release profile can be maintained before and after injection to the host, for therapeutic purpose.

### Example 4 the Influence of Ionic Crosslinker Concentration to the Storage Modulus and Loss Modulus

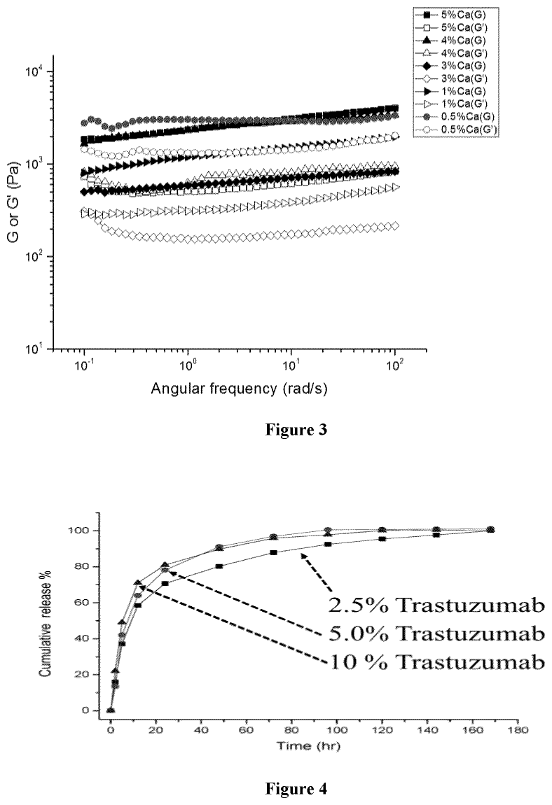

The influence of ionic crosslinker concentration, taking CaCl₂ or CaCO₃ as one exemplary case, on the mechanical property of the nanocomposite gels without the presence of amphiphilic alginate nanoparticles, i.e., AGO2.0 composition, is given in FIG. 3. Both storage modulus where the gel deformed elastically and loss modulus where the gel deformed plastically, show the higher G value for 0.5%, 4% and 5% crosslinker concentrations, suggesting higher rigidity of the gel, however, a large decrease in G′ for higher crosslinker concentrations suggests ease of structural disruption for higher crosslinking gel and in the meantime, we found higher crosslinker concentration causes too fast gelation, and made the final gel with structural inhomogeneity afterward. Therefore, a lower concentration of crosslinker is more applicable to this gel in terms of structural behavior for medical uses.

### Example 5 the In-Vitro Drug Release Profile of the trastuzumab Gel

After the trastuzumab gel, with a drug concentration range of 2.5 wt %, 5 wt %, and 10 wt % (based on gel weight) was prepared, the drug-carrying gels were subjected to in-vitro drug release study, FIG. 4, carried out at ambient environment and in PBS with a solution pH 7.4 and a liquid medium volume three times the volume of the gels for the drug releasing test. Trastuzumab was released reaching 90% at 48-h test, and slow in releasing profile till 7-day period, near 100% of the drug being released out. The releasing rate is apparently faster for the gel with higher trastuzumab, but the drug releasing profile is comparably with each composition, indicating the dominant mechanism of drug release remained similar, regardless drug concentration. However, even though the releasing profile revealed a rapid elution behavior in-vitro in an early-phase of release, we do expect a much slower profile can be achieved since the test condition in-vitro is rather different from that of in-vivo or clinical condition, for instance, subcutaneous environment. Besides, the degradation of the gel itself should also play a role in the resulting releasing profile, and this is likely to be collectively considered as a whole in the release profile given in FIG. 4.

### Example 6 Cytotoxicity Study of the trastuzumab Gel

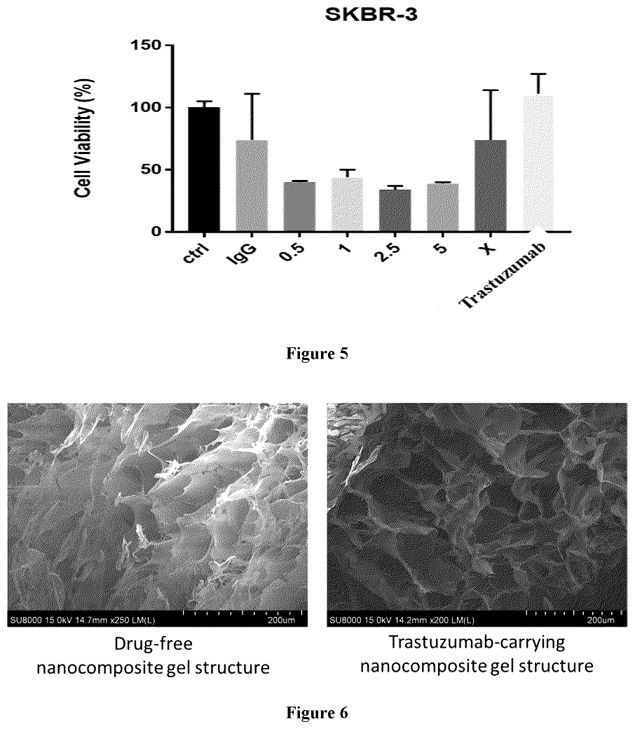

Highly malignant breast cancerous SKBR3 cells were treated with Trastuzumab gel with drug concentration range of 0.5%, 1.0%, 2.5%, and 5%, respectively and respective controls, i.e., positive control and IgG negative control, as indicated in FIG. 5, for 72 h. SKBR3 cells were subjected to MTT assay for analyzing cell survival. Free trastuzumab has 6.25 mg/mL for comparison. Data confirmed efficacy of the Trastuzumab gel.

### Example 7 the Structure of the Nanocomposite Gels

The nanocomposite gel, with and without carrying T-mAb show a highly porous structure after freeze-dried as shown in FIG. 6. The pore size of the gel network is ranging from 30 to 150 micrometers, which is relatively large and is surely facilitating the drug elution. Considering the gel composition, water is taking a very large part of the gel volume, say 85%-95% in volume, and it is reasonable to leave a large porous structure after water was completely removed under freeze-drying condition, while the solid network can be preserved without significant disruption or collapse in structure during drying process, for both drug-free and T-mAb-carrying gels. Such porous gel network also ensures a potential advantage of degradation in a controllable manner, depending on the solid content in the gel product. This will then be a critical variant upon practical uses, especially for consecutive dosing over in-vivo and clinical practices.

### Example 8 the Biosafety of the Nanocomposite Gel

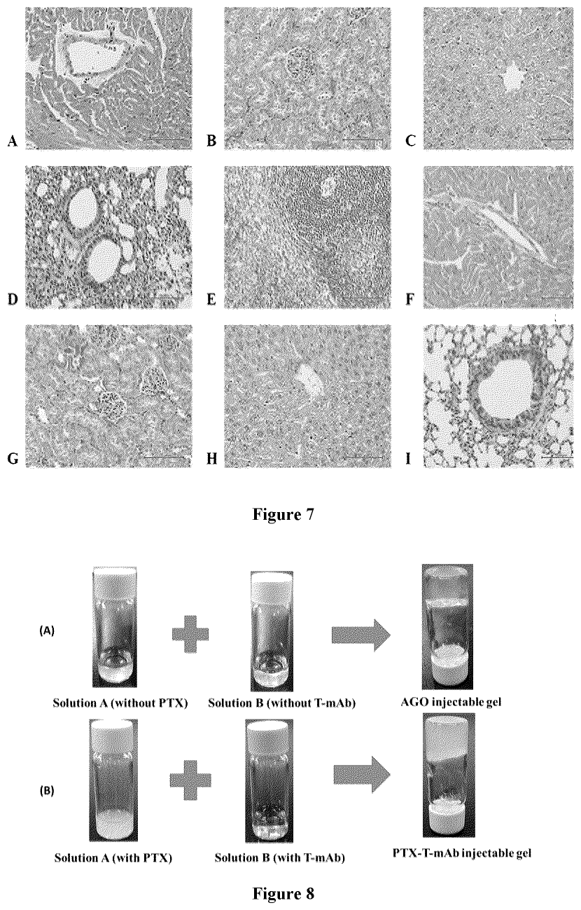

Acute toxicity of the drug-free injectable nanocomposite gel was carried out using ICR mice (n=10) for a time duration of 14 days. The gels with both AGO1.7 and AGO2.0 compositions were injected in an amount of 200 microliter each at subcutaneous site of the right flank region of the mice using a G30 syringe. The weight of the mice was monitored daily and remained constantly increase or similar during the test period. No measurable adverse effect was detected before sacrificed. Histopathological findings of the toxicity study for AGO1.7 and AGO2.0 compositions were examined, as illustrated in FIG. 7. No significant lesion in the heart, kidneys, liver, lungs and spleen was found in the AGO1.7 (A-E) and AGO2.0 (F-I) groups, respectively. (H&E stain. 400×). This finding further evidenced the biosafety of the said nanocomposite gel in this animal model, and in the meantime, the said gel was able to successfully perform a subcutaneous injection practice.

### Example 9 In-Vivo Study of the trastuzumab Gel

The injectable nanocomposite gel carrying biosimilar drug, i.e., trastuzumab, with different dosing concentration designed based on clinical data per dosing, for a subsequent animal study. The breast tumor was cultivated by injection 1×10⁷ SKBR3 cells to the right flank region of the mice, and the controls are given below:
Objects: Seven-week-old BALB/c Nude mice (Female)
Quantity: 5 groups, 4 mice for each group, totally 20.
Drugs: (1) PBS; (2) free-trastuzumab; (3) 1× trastuzumab gel; (4) 2× trastuzumab gel; (5)
3× trastuzumab gel (for 3-week dose at one injection)
Dose: 25 mg/kg and 50 mg/kg, and 75 mg/kg, one SC injection per week
Injection frequency: Three doses on 2 weeks (Subcutaneous injection)
Injection Volume: 100 ul/20 g
Observation: Weight the mice and measure the tumor size twice a week.
Test period: 2-3 weeks, depending on size change of the breast tumor
Injection site: subcutaneous site on the left flank region of the mice
After continue monitoring on the size change of the tumor for on a weekly base, we found the growth of the tumor for the control group (PBS) is significant in the first week, from ˜100 mm³ to nearly 1000 mm³, and for free trastuzumab injection, from ˜100 mm³ to 813 mm³, and for 1× trastuzumab gel, from ˜100 mm³ to 543 mm³, and for 2× trastuzumab gel, from ˜100 mm³ to nearly 410 mm³. And the tumor continued growing for the second week and reach, ˜1800 mm³, ˜1500 mm³, ˜800 mm³, and ˜600 mm³, for PBS, free trastuzumab, 1× trastuzumab gel and 2× trastuzumab gel, respectively.

It is encouraging that albeit the test is failed to effectively reduce or eliminate the tumor significantly for such a HER 2-positive highly-malignant breast tumor. Clinical standard to treat such HER 2-positive breast tumor is using trastuzumab drug or Herceptin®, via SC injection or vein injection, this invention disclosed a new opportunity to use trastuzumab gel where an enhanced therapeutic performance in inhibiting the growth of SKBR-3-derived tumor can be clearly observed, improved by a factor of 2-3 times the size change during the test period, comparing to both control group and free-trastuzumab group. This suggests the use of trastuzumab gel disclosed in this invention improved therapeutic efficacy to a considerable extent, and is worthy of moving toward a potential clinical translation for further application.

### Example 10 Preparation Procedures of AGO Injectable Gels

Two AGO-based nanocomposite injectable gels were prepared as illustrated in FIG. 8, where samples (A) and (B) were successfully made. Sample (A) was prepared following the AGO preparation procedure described in Example 1, over which Solution A and Solution B were prepared separately and mixed to form a clear AGO nanocomposite gel, while the Sample (B) was prepared by first encapsulating paclitaxel (PTX) drug into AGO nanoparticles and mixed with other important ingredients (as that used for Solution A), to form Solution A (with PTX), while Solution B (with T-mAb) was prepared by mixing and dissolving T-mAb with other gel forming ingredients (as that used for Solution B), to form final gel-forming Solution B (with T-mAb). Mixing both solutions: Solution A (with PTX) and Solution B (with T-mAb), a final PTX-T-mAb injectable gel was successfully prepared for a subsequent studied.

### Example 11 In-Vitro Study of the Paclitaxel-Trastuzumab Gel

In-vitro cell viability test was carried out using free paclitaxel and PTX-T-mAb gel over a cell culture condition given as:

### 1. Concentration:

Paclitaxel: T-mAb=1,000: 100 ug/mL, in solution form, as of “Free PTX” and in gel form, as of “PTX gel”, which was prepared according to Sample (B) described in Example 10)
   2. Time: 72 hours
   3. Cell line: SKBR3, 5×104 cells/well (24 well)
   4. Gel volume: 50 μl
   5. Medium volume: 500 μl
   Paclitaxel: T-mAb=1,000: 100 ug/mL, in solution form, as of “Free PTX” and in gel form, as of “PTX gel”, which was prepared according to Sample (B) described in Example 10)

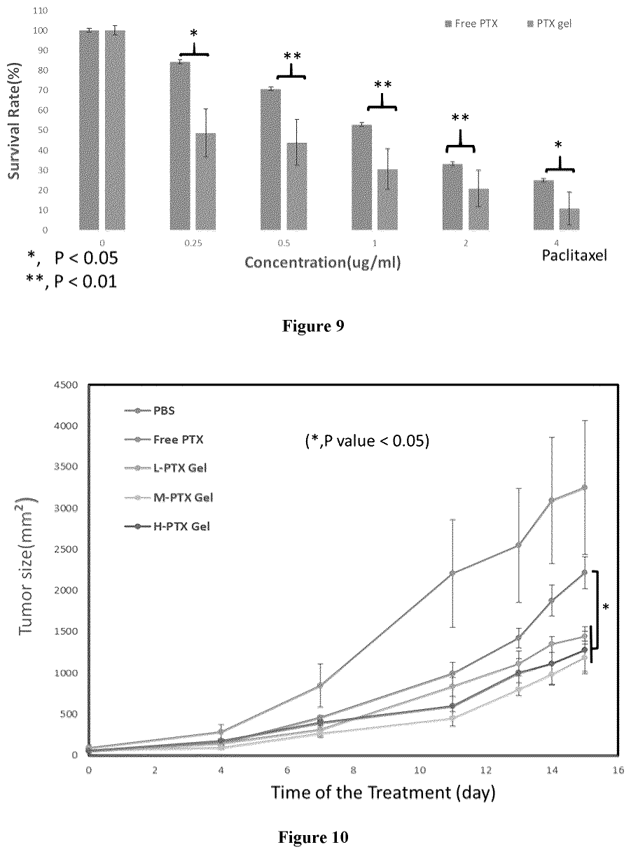

The resulting cell viability is given in FIG. 9, where a considerable cell killing capability can be detected in terms of “PTX gel” sample, while comparing with free paclitaxel. This study ensures the presence of two drugs, both chemo-drug and antibody drugs, encapsulated into AGO-based gel showing a much improved cancerous cell-killing capability, compared with free drug from. The plausible explanation is due to improved solubility of paclitaxel while encapsulated into the AGO nanoparticles, to form a final gel structure. The encapsulated paclitaxel appeared to show an improved cell availability, while the free paclitaxel (in precipitated form in the culture medium) showed poor cell availability, to kill SKBR-3 cell.

### Example 12 In-Vivo Study of the Paclitaxel-Trastuzumab Gel

The injectable nanocomposite gel carrying both chemo-drug, i.e., paclitaxel, and biosimilar drug, i.e., trastuzumab (T-mAb), with different dosing concentrations designed based on clinical data per dosing, for a subsequent animal study. The breast tumor was cultivated by injection 1×10⁷ SKBR3 cells to the right flank region of the mice, and the controls are given below:
Objects: Seven-week-old BALB/c Nude mice (Female)
Quantity: 5 groups, 4 mice for each group, totally 20.
Drugs: (1) PBS; (2) free-Paclitaxl: T-mAb; (3) 1× Paclitaxel: T-mAb gel (as of “L-PTX gel”); (4) 2× Paclitaxel: T-mAb gel (as of “M-PTX gel”); (5) 3× Paclitaxel: T-mAb gel (as of “H-PTX gel).
Dose: Paclitaxel: T-mAb=10:1
Injection frequency: Three doses on 2 weeks (Subcutaneous injection)
Injection Volume: 100 ul/20 g
Observation: measure the tumor size regularly.
Test period: 2-3 weeks, depending on size change of the breast tumor
Injection site: subcutaneous site on the left flank region of the mice

The resulting tumor size measurement over the time duration (15-day duration) of animal study is illustrated in FIG. 10. It is clearly to show that the use of M-PTX and H-PTX injectable gels enabled a considerable therapeutic potency (efficacy) in inhibiting the growth of SKBR-3 derived tumor, compared with other experimental controls, especially the one with free drug combination, i.e., termed as “Free PTX” (P<0.05).

This study also indicates a sustain release of both water-insoluble chemo-drug, paclitaxel, and water-soluble, antibody drug T-mAb, that can be co-delivered effectively against highly-metastasized HER2-positive breast tumor with synergy, compared with co-administration of both drugs in their free form.

It is encouraging that a co-delivery and co-release of anti-breast tumor drugs of distinct physico-chemical and therapeutic properties through the use of injectable AGO-based gel for SC administration can be technically and therapeutically achieved in the prevention of metastasized HER2-positive breast tumor from substantiated growth in animal body.

Clinical standard to treat such HER 2-positive breast tumor is typically using trastuzumab drug or Herceptin®, or a combination therapy of T-mAb and chemo-drug administrated in sequential manner via mostly vein injection or some via SC injection, this invention disclosed a new opportunity to use AGO-based injectable gel to carry a single high-dose Biosimilar drug or a combination of Biosimilar drug, i.e., T-mAb, and a traditional chemo-drug, i.e., paclitaxel, followed by co-releasing both drugs from the gel where an enhanced therapeutic performance in inhibiting the growth of SKBR-3-derived tumor, as model tumor, can be clearly observed, improved by a factor of 2-4 times the tumor size change during the test period, comparing to both control group and free-T-mAb group. This suggests the use of AGO-based nanocomposite gel disclosed in this invention improved therapeutic efficacy to a considerable extent, and is worthy of moving toward a potential clinical translation for further anti-cancer application.

All patents and references cited in this specification are incorporated herein in their entirety as reference. Where there is conflict, the descriptions in this case, including the definitions, shall prevail.

While the invention has been described in connection with what are considered the exemplary embodiments, it is understood that this invention is not limited to the disclosed embodiments but is intended to cover various arrangements included within the spirit and scope of the broadest interpretation so as to encompass all such modifications and equivalent arrangements.

1-32. (canceled)

33. A composition of injectable nanocomposite gel, which comprises:
an amphiphilic alginate nanoparticle, wherein the amphiphilic alginate nanoparticle is an oleic acid-conjugated alginate and wherein the amphiphilic alginate nanoparticle has a molecular weight of 5,000 g/mole to 50,000 g/mole;
a hyaluronic salt or derivative, wherein the hyaluronic salt has a molecular weight of 100,000 g/mole to 1,000,000 g/mole, preferably 100,000 g/mole to 500,000 g/mole;
an alginate salt or derivative, wherein the alginate salt is sodium alginate and has a molecular weight of 10,000 g/mole to 60,000 g/mole; and
a mixture of ionic crosslinkers, wherein the mixture of ionic crosslinkers comprise two or more ionic crosslinkers, and wherein the ionic crosslinkers contain one more divalent or trivalent cation, and wherein the ionic crosslinkers is a chloride salt or a carbonate salt or a phosphate salt, and wherein the gross concentration of the ionic crosslinker is from 0.5% to 5% (on gel weight base).

34. The composition of claim 33, further comprising an active ingredient.

35. The composition of claim 34, wherein the active ingredient is selected from the group consisting of an antibody drug, a biosimilar drug, a protein-like drug, a chemo-drug, and the combination thereof.

36. The composition of claim 34, wherein the fatty acid-conjugated alginate is selected from the group consisting of oleic acid-conjugated alginate, stearic acid-conjugated alginate, linoleic acid-conjugated alginate, palmitic acid-conjugated alginate, and the combinations thereof.

37. The composition of claim 34, wherein the active ingredient is selected from the group consisting of trastuzumab, bevacizumab, gemtuzumab, inotuzumab, polatuzumab, sacituzumab, adalimumab, infliximab, rituximab, and the combinations thereof.

38. The composition of claim 34, wherein the active ingredient is a water-insoluble active ingredient.

39. The composition of claim 38, wherein the water-insoluble active ingredient is selected from the group consisting of vitamin A and its derivatives, Vitamin E and its derivatives, paclitaxel, docetaxol, camptothecin, doxorubicine, and curcumin.

40. The composition of claim 33, wherein the ionic crosslinker is selected from the group consisting of AlCl₃, CaCl₂), CaCO₃, calcium phosphates, ZnCl₂, BaCl₂.

41. The composition of claim 33, wherein the injectable nanocomposite gel contain highly porous structure, wherein the highly porous structure has a pore size of 30 to 150 micrometers.

42. A method for preparing the composition set forth in claim 33, comprising the steps of:
(1) preparing a mixture of alginate-based solution as Solution (1), comprising an alginate salt, where the alginate salt is sodium alginate and has a molecular weight of 10,000 g/mole to 60,000 g/mole, and an amphiphilic alginate nanoparticle, wherein the amphiphilic alginate nanoparticle is oleic acid-conjugated alginate and wherein the amphiphilic alginate nanoparticle has a molecular weight of 5,000 g/mole to 50,000 g/mole, at the ratio ranging from 1:1 to 10:1;
(2) mixing a hyaluronate, wherein the hyaluronate is hyaluronic salts and has a molecular weight of 100,000 g/mole to 1,000,000 g/mole, preferably 100,000 g/mole to 500,000 g/mole, with a mixture of ionic crosslinkers, wherein the mixture of ionic crosslinkers comprise two or more ionic crosslinkers, and wherein the ionic crosslinkers contain one more divalent or trivalent cation, and wherein the ionic crosslinkers is a chloride salt or a carbonate salt or a phosphate salt, and wherein the gross concentration of the ionic crosslinker is from 0.5% to 5% (on gel weight base), to obtain a mixture as Solution (2);
(3) mixing Solution (1) and Solution (2) at the ratio (by weight) ranging from 0.5:1 to 5:1 to obtain a homogeneous nanocomposite gel.

43. The method of claim 42, wherein the ratio of Solution (1) to Solution (2) ranges from 0.5:1 to 2:1

44. The method of claim 42, wherein an active ingredient is encapsulated into the homogeneous nanocomposite gel, comprising following steps:
(1) dissolving the active ingredient and mixed in Solution (1) at the concentration ranging (on the gel weight base) from 1.0% to 15% by weight to form Solution (3);
(2) mixing Solution (2) and Solution (3) under continuous stirring to obtain a solid-like injectable drug-carrying gel.

45. The method of claim 44, wherein the active ingredient is a biosimilar or an antibody drug.

46. The method of claim 42, wherein the amphiphilic alginate nanoparticle further comprises an active ingredient.

47. The method of claim 42, wherein the active ingredient is a water-insoluble active ingredient.

48. The method of claim 47, wherein the water-insoluble active ingredient is selected from the group consisting of vitamin A and its derivatives, Vitamin E and its derivatives, paclitaxel, docetaxol, camptothecin, doxorubicine, and curcumin.

49. The method of claim 42, wherein the ionic crosslinker is selected from a group consisting of the group consisting of AlCl₃, CaCl₂), CaCO₃, calcium phosphates, ZnCl₂, or BaCl₂.

50. The method of claim 43, wherein the biosimilar or the antibody drug is selected from the group consisting of trastuzumab, bevacizumab, gemtuzumab, inotuzumab, polatuzumab, sacituzumab, adalimumab, infliximab, rituximab, and the combinations thereof.

51. The method of claim 43, wherein the concentration of the antibody or the biosimilar drug to be encapsulated and delivered is ranging from 1.0% to 15% in weight (on gel weight base).

52. The composition of claim 33, wherein the composition is an injectable gel is administered via subcutaneous injection, intramuscular injection, intratumoral injection, or intraperitoneal injection for anticancer treatment.
